(19) United States
(12) Patent Application Publication
(10) Pub. No.: US 2014/0162352 A1
(43) Pub. Date: Jun. 12, 2014

(54) Dual Use Petri Plate With Locking Feature

(71) Applicant: PHOENIX BIOMEDICAL PRODUCTS INC., Mississauga (CA)

(72) Inventors: Ezzat Iskander, Caledon East (CA); Andre Iskander, Mississauga (CA)

(73) Assignee: Phoenix Biomedical Products Inc., Mississauga (CA)

(21) Appl. No.: 13/856,176

(22) Filed: Apr. 3, 2013

Related U.S. Application Data
(60) Provisional application No. 61/735,020, filed on Dec. 9, 2012.

(51) Int. Cl. C12M 1/22

(52) U.S. Cl. CPC C12M 23/10
USPC 435/305.4; 29/525

(57) ABSTRACT

A dish structure includes: (a) a bottom container having an outer cylindrical wall and a diametric base interconnecting the outer cylindrical wall; (b) a lid having an inverted cylinder provided with a downwardly extending cylindrical wall, the lid being sized so that the downwardly extending cylindrical wall associates with the outer cylindrical wall of the bottom container; (c) a frictionally-engagable member integral with the surface of the downwardly extending cylindrical wall of the lid; (d) a frictionally engagable member integral with the surface of the wall of the bottom container; and (e) at least one of the bottom container and the lid being formed of flexibly deformable synthetic plastic material, which is thin enough to have the characteristics of being flexibly deformable. By such structure the lid and bottom container base may remain loosely assembled (unlocked), or by user manipulation be tightly assembled into a reversibly locked configuration.

### TECHNICAL FIELD

The description relates to members of the Petri dish/plate family including the contact plate.

### BACKGROUND

One member of the Petri dish/plate family is the conventional Petri dish. Petri dishes are well known and in common usage, particularly in laboratories for producing cultures in a culture medium such as agar. Such dishes consist of a lid and a base, usually with a flat bottom and short cylindrical sides.

The Petri dish is commonly used for producing microbial cultures in a culture medium such as agar. The culture medium is in the bottom of the Petri dish and the Petri dish lid sits loosely on the bottom to prevent contamination. In this conventional design, the Petri dish lid is susceptible to separation from the bottom.

The contact plate is another member of the Petri dish/plate family. A contact plate facilitates easy and reproducible surface microbial testing and monitoring of the microbial flora on environmental surfaces. Surface sampling is achieved by gently rolling the domed surface of the agar onto the area to be tested. A grid on the base of the plate subdivides the growth areas to aid accurate colony counts.

Contact plates are used in conjunction with diagnostic culture media as a sampling device. The base of a contact plate is filled with media in such a way that the media forms a positive meniscus which projects above the outer rim of the base. This allows the user to hold the base and use the projecting media surface to take direct samples by touching the media to the desired surface. Due to the outward projection of the media, which facilitates easy sampling, the contact plate is vulnerable to contamination. Conventional design contact plates utilize a loosely fitting lid that simply sits upon the base as a cover for the media.

### SUMMARY

In some general aspects, a structure includes a lid and a base structure that allows for the lid to remain loosely assembled or to be tightly assembled into a locked configuration. The structure also enables the lid to be used as part of a member of the Petri dish/plate family, in which the lid of the Petri dish/plate can remain loosely assembled or be tightly assembled into a locked configuration.

In some general aspects, a Petri dish/plate is provided that can be in a closed and unlocked configuration, or can easily and simply be manipulated to be in a locked configuration.

While in the closed but unlocked configuration, the lid is oriented concentrically with the base below, and is loosely resting upon the base in a manner similar to conventional Petri dish/plates. This is achieved through an interference design, for example, a projecting circumferential ring integrally associated with at least one of the two components at their mating surfaces. In more general terms the interference feature(s) can be in the form of a lip or ring, or a portion of a lip or ring, or other feature, projecting either inwardly or outwardly from the circumference of the lid, and inwardly or outwardly from the circumference of the base. Such portions of features can be small enough so that they can essentially be considered individual projections or cavities (ribs, dots or dimples). These features do not allow the locking of the lid to the base under normal handling conditions, and keep the lid reliably positioned when not in the tightest (locked) assembly condition.

The locked state of the lid to the base is achieved when sufficient force has been applied to press together the loosely assembled components. The lid, base, or both are preferably formed of flexibly deformable synthetic plastic material, for example polystyrene, polyethylene or polypropylene or polyvinyl chloride, which is thin enough to have the characteristics of being flexibly deformable so that the lid or base slip or deform to a degree that allows the lid to travel past or into those features and become trapped or locked to the base. Those features described above as interference features, in addition to other features, can also act as the locking features, essentially locking the lid to the base. This lock can be released by deforming the base and/or lid and/or other release feature integrated within the parts to a degree that allows the locking feature(s) to disengage. Once unlocked, the lid and base can then be left assembled in the loose configuration, be disassembled, or be again locked.

In another implementation, the Petri dish/plate configuration requires no special orientation, alignment, rotation nor any special positioning in order to utilize the locking feature. The base and/or the lid of the Petri dish/plate is also preferably formed of flexibly deformable synthetic plastic material, for example polystyrene, polyethylene, polypropylene or polyvinyl chloride, which is thin enough to have the characteristics of being flexibly deformable. The lid need only be placed concentrically on top of the base. It is then up to the user's discretion to press the lid into the locked state. The Petri dish/plate can be used as a conventional Petri dish/plate when the locking feature is not utilized, or can also be used as a locking Petri dish/plate. This is a dual purpose Petri dish/plate. Whereas other variations of Petri dish/plates can lack a locking mechanism, lack a loose configuration, or require special orientation to lock, this Petri dish/plate inherently offers easily usable and unique features. It is the user's decision how to utilize this Petri dish/plate.

Thus, a Petri dish/plate is provided in which the upper cover (lid) and/or the bottom container (base) are outfitted with the above-described unique physical features such that the lid and base can remain loosely assembled (unlocked), or by user manipulation be tightly assembled into a locked configuration. The value of a locking mechanism is to prevent accidental exposure and ensure integrity of the medium after inoculation, during incubation and transportation, and therefore protect from contamination.

In some general aspects, a member of the Petri dish/plate family is provided comprising (a) a bottom container, the bottom container having a cylindrical wall and a diametric base interconnecting the cylindrical wall; (b) a lid comprising an inverted cylinder provided with a downwardly extending cylindrical wall, the lid being sized so that the lid wall associates loosely with the wall of the bottom container; (c) a frictionally engagable member integral with at least one of the inner or outer surface of the lid wall; (d) a frictionally engagable member integral with at least one of the inner or outer surface of the bottom container wall; and (e) at least one of the bottom container and the lid being formed of flexibly deformable synthetic plastic material, which is thin enough to have the characteristics of being flexibly deformable.

In other general aspects, a member of the Petri dish/plate family is provided comprising (a) a bottom container, the bottom container having an outer downwardly extending cylindrical wall, a diametric base interconnecting between the outer downwardly extending cylindrical wall and a diametrically-spaced inner cylindrical wall extending upwardly from the diametric base; (b) a lid comprising an inverted cylinder provided with a downwardly extending cylindrical wall, the lid being sized so that the lid wall fits between the outer wall and the inner wall of the bottom container; (c) a frictionally engagable member integral with the outer surface of the lid wall; (d) a frictionally engagable member integral with the inner surface of the outer wall of the bottom container; and (e) at least one of the bottom container and the lid being formed of flexibly deformable synthetic plastic material, which is thin enough to have the characteristics of being flexibly deformable.

In other general aspects, a member of the Petri dish/plate family is provided comprising (a) a bottom container, the bottom container having an outer cylindrical sidewall and a diametric base interconnecting the cylindrical sidewall; (b) a lid comprising an inverted cylinder provided with a downwardly extending cylindrical wall, the lid being sized so that the lid wall fits outside the sidewall of the bottom container; (c) a frictionally engagable member integral with the inner surface of the lid wall; (d) a frictionally engagable member integral with the outer surface of the sidewall of the bottom container; and (e) at least one of the bottom container and the lid being formed of flexibly deformable synthetic plastic material, which is thin enough to have the characteristics of being flexibly deformable.

In other general aspects, a method is provided by the steps of providing a member of the Petri dish/plate family interchangeably in either loose assembly, or a tight/locked assembly. The method includes providing a member of the Petri dish/plate family comprising a bottom container, the bottom container having a cylindrical wall and a diametric base interconnecting the cylindrical wall; a lid comprising an inverted cylinder provided with a downwardly extending cylindrical wall, the lid being sized so that the lid wall associates with the wall of the bottom container, the lid including a projection that is integral with and projects from the lid wall; a frictionally engagable member integral with at least one of the inner or outer surface of the lid wall; and a frictionally engagable member integral with at least one of the inner or outer surface of the bottom container wall; and at least one of the bottom container and the lid being formed of flexibly deformable synthetic plastic material, which is thin enough to have the characteristics of being flexibly deformable. The method includes placing the lid over the bottom container so that the downwardly extending cylindrical wall of the lid associates with the cylindrical wall of the bottom container, whereby the lid rests freely on the bottom container due to the interference feature of the frictionally-engagable member not yet being engaged, and so that the lid remains properly located conventionally on the bottom container; and manually applying a compressive force which is sufficient to deform the lid or base or both, to a degree that the frictionally engagable member of the lid wall engages the frictionally engagable member of the bottom container wall and become locked, whereby the lid is trapped and reversibly locked to the base.

The described structure and design allows the lid and the base of the members of the Petri dish/plate family to be loosely assembled and allows the lid to be reversibly locked in place to the base by a simple manipulation that does not require a specific orientation, alignment, rotation or any special positioning of the lid on the base.

### DESCRIPTION OF THE DRAWINGS

FIG. 1 shows a perspective view, a side view, and a close-up side view of a cross-section of the lid and base of a contact plate with a lip and a ring integrated into the lid and bottom container.

FIG. 2A is a cross-sectional side view of a contact plate showing the opened configuration.

FIG. 2B is a cross-sectional side view of the contact plate showing the loosely assembled configuration.

FIG. 2C is a cross-sectional side view of the contact plate showing the tight/locked configuration.

FIG. 3A shows a perspective view, a side view, and a close-up side view of a cross-section of the contact plate, showing the loosely assembled configuration.

FIG. 3B shows a perspective view, a side view, and a close-up side view of a cross-section of the contact plate, showing the tight/locked configuration.

FIG. 4 shows a perspective view, a side view, and a close-up side view of a cross-section of the lid and base of a Petri dish with a rib integrated in the lid and a lip integrated in the base.

FIG. 5A shows a perspective view, a side view, and a close-up side view of a cross-section of a Petri dish with ventilation lugs showing the loose assembled configuration.

FIG. 5B shows a perspective view, a side view, and a close-up side view of a cross-section of the Petri dish with ventilation lugs showing the tight/locked configuration.

FIG. 6A shows a cross-sectional view of a Petri dish with ventilation lugs showing the opened configuration.

FIG. 6B shows a cross-sectional view of a Petri dish with ventilation lugs showing the loose assembled configuration.

FIG. 6C shows a cross-sectional of a Petri dish with ventilation lugs showing the tight/locked configuration.

### DETAILED DESCRIPTION

### Example 1

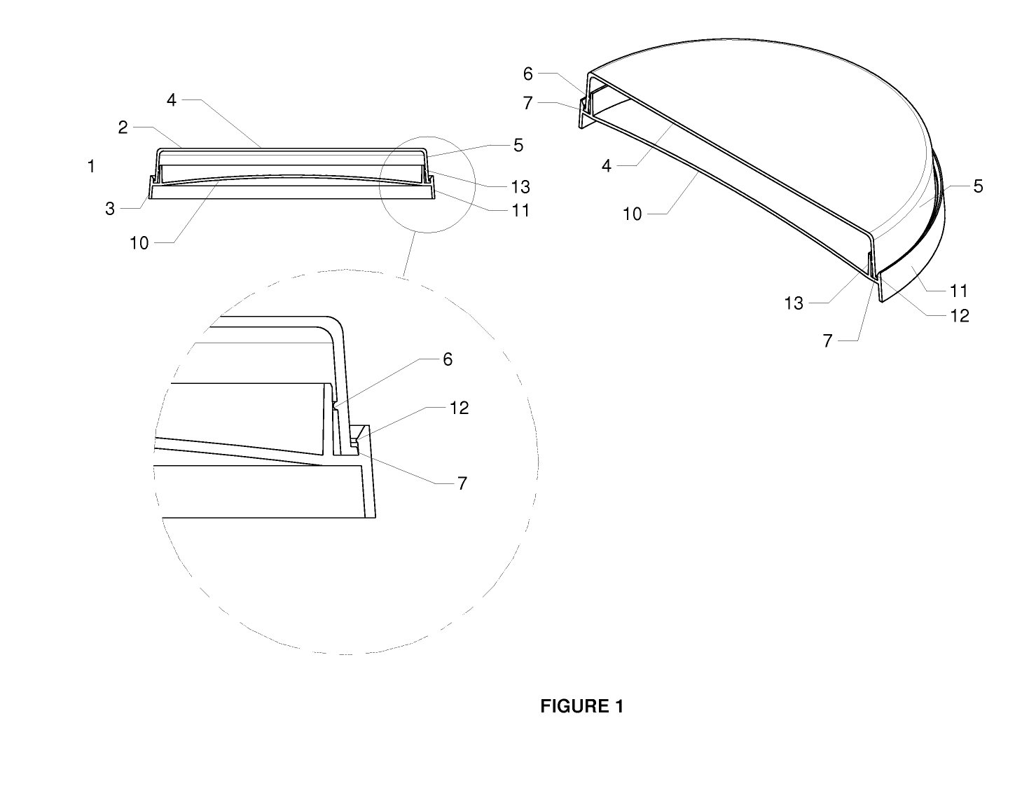

Referring to FIGS. 1 to 3B, a contact plate 1 has a contact plate lid 2 and a contact plate base 3. While a contact plate has been shown in this implementation, another implementation of similar structure is a Petri dish, described in Example 2.

The lid 2 has a substantially flat horizontal top portion 4 and a short substantially cylindrical side wall 5 integral with and around the top portion 4. A ring 6 projects inwardly from and encircles the inner circumferential face of the side wall 5. A lip 7 is integral with and projects outwardly from the bottom edge of the side wall 5.

The contact plate base 3 has a horizontal bottom portion 10 and a cylindrical outer side wall 11 encircling the bottom portion 10 and extending vertically both above and below the bottom portion 10. A lip 12 is integral with and projects inwardly from the top edge of the side wall 11. An inner cylindrical side wall 13 extends vertically upwardly and a short diametric distance from the outer circumference of the bottom portion 10.

While this FIGS. 1 to 3B show that the ring 6 projects inwardly from the inner circumferential face of the side wall 5 of the lid 2, in another implementations, a ring can be provided, the ring projecting outwardly from the outer circumferential face of the side wall 13 of the base 3.

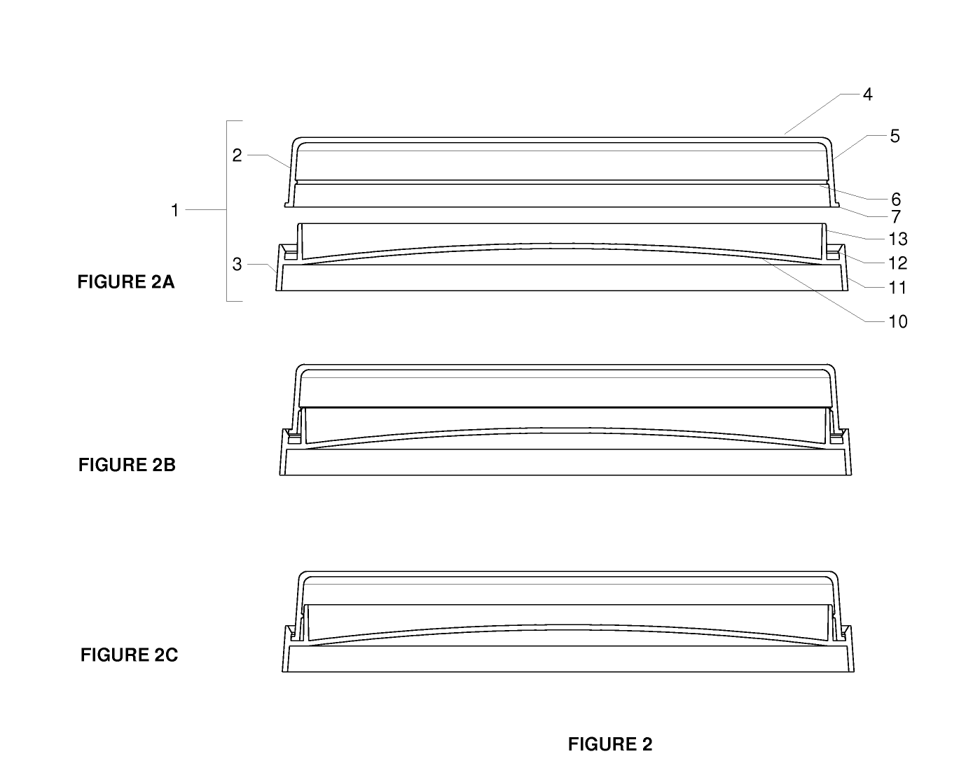

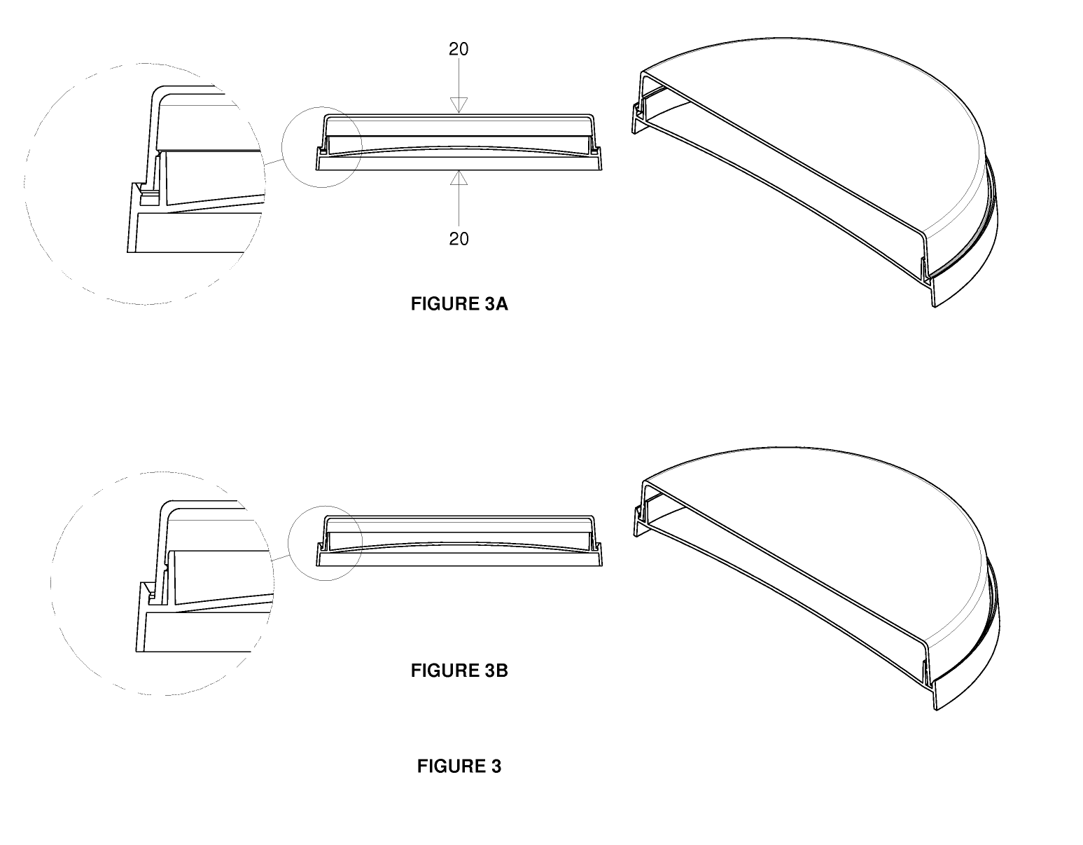

In operation, as shown in FIGS. 2A-3B, the contact plate 1 can be in the opened state, wherein the lid 2 is completely separate from the base 3, as shown in FIG. 2A.

The contact plate can also be in a loose assembled configuration, wherein the lid 2 rests freely on the base 3 due to interference features of the side walls 5, 11 and 13, as shown in FIG. 2B. In this state, the locking features have not yet been engaged and the lid remains properly located on the base as a conventional contact plate would.

Finally, the contact plate can be in a tight/locked assembled configuration, shown in FIG. 2C, wherein the lid is trapped in position by the inner ring 6, the outer lip 7 and the inner lip 12. This state is not permanent and may be released by deforming the lid or base or both.

The operation of the contact plate 1 is further illustrated in FIGS. 3A and 3B. In FIG. 3A, the lid 2 rests above the locking features and is in the loosely assembled configuration. When the user applies compressive force 20 sufficient to deform the lid or base or both, to a degree that the inner ring 6, the lip 7 and the lip 12 slip and become locked, the lid 2 is trapped and reversibly locked to the base 3 as shown in FIG. 3B.

Thus, in summary, in the loose assembly, the lid rests freely on the base due to the interference feature of the frictionally-engagable ring. Locking has not yet been engaged and the lid remains properly located conventionally on the base.

In the tight/locked assembly. in order to lock, a compressive force is applied which is sufficient to deform either or both of the lid or the base so that the locking features slip and become locked. The lid is thus trapped and locked to the base.

However, the lid is removably trapped in position by the interference features. To unlock, a compressive force is applied which is sufficient to deform either or both of the lid or base so that the locking features slip and become unlocked. Consequently, release is achieved by deforming either or both of the lid and base.

### Example 2

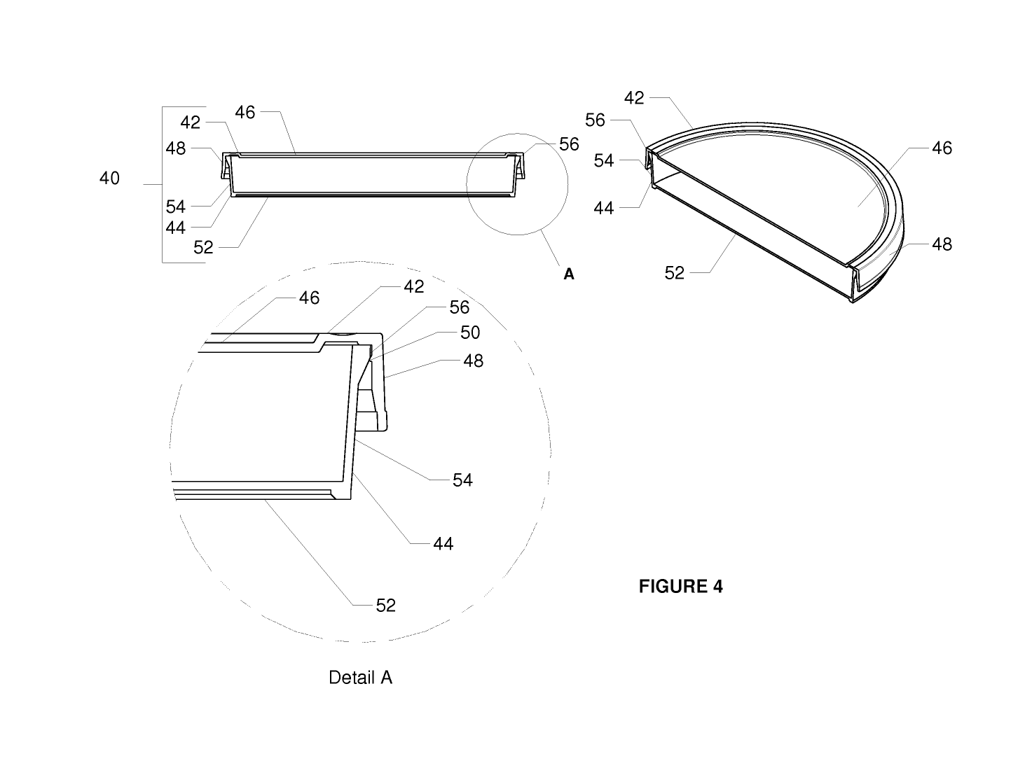

In another implementations, as shown in FIGS. 4 to 6C, a petri dish 40 has a petri dish lid 42 and a petri dish base 44.

As shown in FIG. 4, the lid 42 has a substantially flat horizontal top portion 46 and a short substantially cylindrical side wall 48 integral with and around the top portion 46. Ribs 50 project inwardly from the inner face of the side wall 48.

The petri dish base 44 has a horizontal bottom portion 52 and a cylindrical outer side wall 54 encircling the bottom portion and extending vertically above the bottom portion. A lip 56 is integral with and projects outwardly from the top edge of the side wall 54.

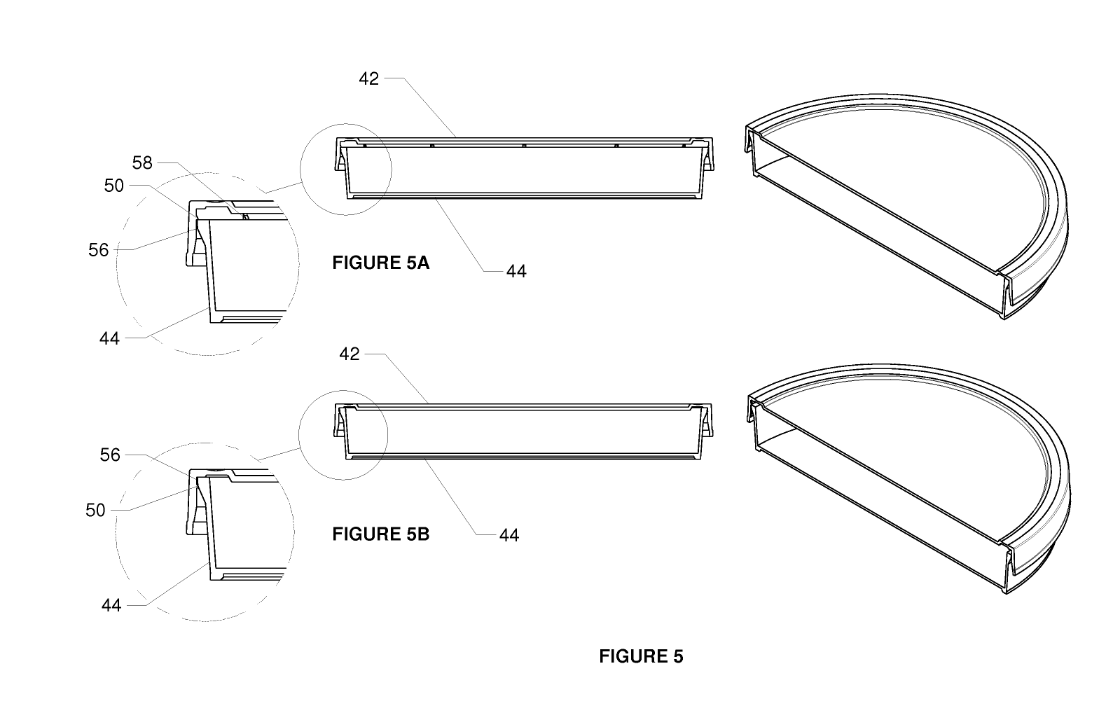

In another implementation, as shown in FIGS. 5A and 5B, ventilation lugs 58 are provided, the lugs 58 being openings that project vertically from the inner face of the top portion of the lid, as shown in FIG. 5A. The petri dish 40 can be in the loosely assembled state, wherein the lid 42 rests freely on the base 44 due to interference features of the ribs 50 and the lip 56 on the side wall 54 of the base 44. The petri dish can be in a tight/locked assembled configuration, shown in FIG. 5B, wherein the lid 42 is trapped in position by the ribs 50 and the lip 56 on the side wall 54 of the base 44. The ventilation lugs 58 allow air ventilation into the loosely assembled and the tight/locked assembled petri dish.

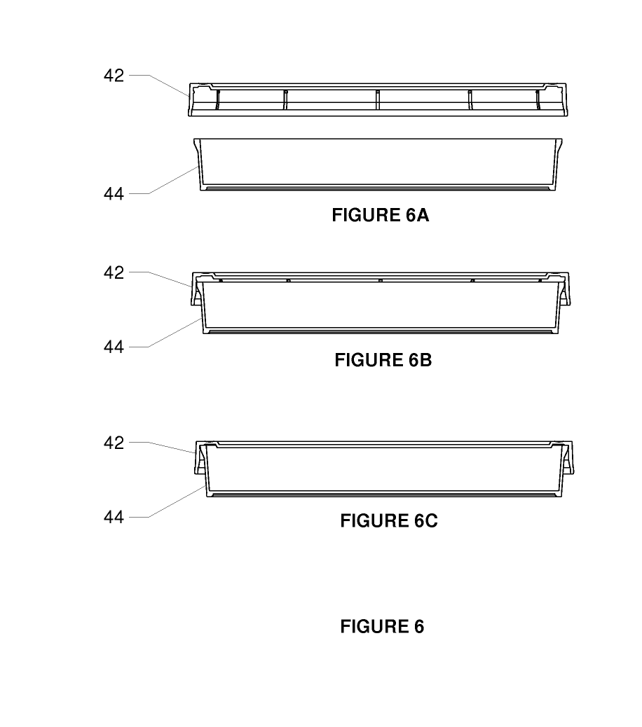

In operation, shown in FIGS. 6A-6C, the petri dish 40 can be in the opened state, wherein the lid 42 is completely separate from the base 44, as shown in FIG. 6A.

The petri dish can also be in a loose assembled configuration, wherein the lid 42 rests freely on the base 44 due to interference features of the ribs 50 and the lip 56 on the side wall 54 of the base 44, as shown in FIG. 6B. In this state, the locking features have not yet been engaged and the lid remains properly located on the base as a conventional petri dish would.

Finally, the petri dish can be in a tight/locked assembled configuration, shown in FIG. 6C, wherein the lid 42 is trapped in position by the ribs 50 and the lip 56 on the side wall 54 of the base 44. This state is achieved by applying a compressive force sufficient to deform the lid 42 or base 44 or both, to a degree that the ribs 50 and the lip 56 slip and become locked, so the lid 42 is trapped and reversibly locked to the base 44. The tight/locked assembled configuration is not permanent and can be released by deforming the lid or base or both.

Thus, in summary, in the loose assembly, the lid rests freely on the base due to the interference feature of the frictionally-engagable ribs. Locking has not yet been engaged and the lid remains properly located conventionally on the base.

In the tight/locked assembly. in order to lock, a compressive force is applied which is sufficient to deform either or both of the lid or the base so that the locking features slip and become locked. The lid is thus trapped and locked to the base.

However, the lid is removably trapped in position by the interference features. To unlock, a compressive force is applied, the compressive force being sufficient to deform either or both of the lid or base so that the locking features slip and become unlocked. Consequently, release is achieved by deforming either or both of the lid and base.

Other implementations are within the scope of the following claims. For example, the dual use locking lid and base could have applications in other types of cylindrical containers that comprise a lid and base.

What is claimed is:

1. A dish structure comprising:
a bottom container, the bottom container having a cylindrical wall and a diametric base interconnecting the cylindrical wall;
a lid comprising an inverted cylinder provided with a downwardly extending cylindrical wall, the lid being sized so that the lid wall associates loosely with the wall of the bottom container;
a frictionally engagable member integral with at least one of the inner or outer surface of the lid wall;
a frictionally engagable member integral with at least one of the inner or outer surface of the bottom container wall; and
at least one of the bottom container and the lid being formed of flexibly deformable synthetic plastic material that is thin enough to have the characteristics of being flexibly deformable.

2. The dish structure of claim 1, comprising a Petri dish.

3. The dish structure of claim 1, comprising a contact plate.

4. The dish structure of claim 1, wherein the frictionally-engagable member is integral with the inner cylindrical surface of the wall of the lid.

5. The dish structure of claim 1, wherein the frictionally-engagable member is integral with the outer cylindrical surface of the wall of the lid.

6. The dish structure of claim 1, wherein the frictionally-engagable member is integral with the outer cylindrical surface of the wall of the bottom container.

7. The dish structure of claim 1, wherein the frictionally-engagable member is integral with the inner cylindrical surface of the wall of the bottom container.

8. The dish structure of claim 1, wherein the frictionally-engagable member comprises a circular ring, an interrupted circular ring, or a plurality of ribs.

9. The dish structure of claim 1, wherein the flexibly deformable synthetic plastic material comprises polyethylene, polystyrene, polypropylene, or polyvinyl chloride.

10. A dish structure comprising:
a bottom container, the bottom container having an outer cylindrical wall, a diametric base interconnecting between the outer cylindrical wall and a diametrically-spaced inner cylindrical wall extending upwardly from the diametric base;
a lid comprising an inverted cylinder provided with a downwardly extending cylindrical wall, the lid being sized so that the lid wall fits between the outer wall and the inner wall of the bottom container;
a frictionally engagable member integral with the outer surface of the lid wall;
a frictionally engagable member integral with the inner surface of the outer wall of the bottom container; and
at least one of the bottom container and the lid being formed of flexibly deformable synthetic plastic material which is thin enough to have the characteristics of being flexibly deformable.

11. The dish structure of claim 10, wherein the frictionally-engagable member comprises a circular ring, an interrupted circular ring, or a plurality of ribs.

12. The dish structure of claim 10 wherein the lid includes a lip that is integral with and projects outwardly from the bottom edge of the downwardly-extending cylindrical wall.

13. The dish structure of claim 10 wherein the bottom container includes a lip that is integral with and projects inwardly from the top edge of the outer cylindrical wall.

14. The dish structure of claim 10 wherein the flexibly deformable synthetic plastic material comprises polyethylene, polystyrene, polypropylene, polyvinyl chloride.

15. A dish structure comprising:
a bottom container, the bottom container having an outer cylindrical sidewall and a diametric base interconnecting the cylindrical sidewall;
a lid comprising an inverted cylinder provided with a downwardly extending cylindrical wall, the lid being sized so that the lid wall fits outside the sidewall of the bottom container;
a frictionally engagable member integral with the inner surface of the lid wall;
a frictionally engagable member integral with the outer surface of the sidewall of the bottom container; and
at least one of the bottom container and the lid being formed of flexibly deformable synthetic plastic material, which is thin enough to have the characteristics of being flexibly deformable.

16. The dish structure of claim 15, wherein the frictionally-engagable member comprises a circular ring, an interrupted circular ring, or a plurality of ribs.

17. The dish structure of claim 15, wherein the flexibly deformable synthetic plastic material comprises polyethylene, polystyrene, polypropylene, or polyvinyl chloride.

18. A method comprising:
providing a dish structure comprising a bottom container, the bottom container having a cylindrical wall and a diametric base interconnecting the cylindrical wall; a lid comprising an inverted cylinder provided with a downwardly extending cylindrical wall, the lid being sized so that the lid wall associates with the wall of the bottom container, the lid including a projection that is integral with and projects from the lid wall; a frictionally engagable member integral with at least one of the inner or outer surface of the lid wall; and a frictionally engagable member integral with at least one of the inner or outer surface of the bottom container wall; and at least one of the bottom container and the lid being formed of flexibly deformable synthetic plastic material, which is thin enough to have the characteristics of being flexibly deformable;
placing the lid over the bottom container so that the downwardly extending cylindrical wall of the lid associates with the cylindrical wall of the bottom container, whereby the lid rests freely on the bottom container due to the interference feature of the frictionally-engagable member not yet being engaged, and so that the lid remains properly located conventionally on the bottom container; and
manually applying a compressive force that is sufficient to deform the lid or base or both, to a degree that the frictionally engagable member of the lid wall engages the frictionally engagable member of the bottom container wall and become locked, whereby the lid is trapped and reversibly locked to the base.

19. The method of claim 18, further comprising applying a compressive force that is sufficient to deform either or both of the lid or base so that the locking features slip and become unlocked.
